(19) United States
(12) Patent Application Publication
(10) Pub. No.: US 2004/0211421 A1
(43) Pub. Date: Oct. 28, 2004

(54) Air-to-air heat exchange for medical ventilator

(75) Inventor: Terry Blansfield, Orange, CA (US)

Correspondence Address: Kit M. Stetina, STETINA BRUNDA GARRED & BRUCKER, Suite 250, 75 Enterprise, Aliso Viejo, CA, 92656, US

(73) Assignee: Bird Products Corporation, a California Corporation

(21) Appl. No.: 10/370,642

(22) Filed: Feb. 20, 2003

(51) Int. Cl. A61M 37/00

(52) U.S. Cl. 128/204.17

(57) ABSTRACT

An air-to-air heat exchanger for pre-cooling and humidifying a breathing gas supplied to a patient. The air-to-air heat exchanger is made of low specific heat material and has an elongate conduit, a plurality of interior fins and a plurality of exterior fins. The elongate conduit is installed adjacent the output of an inspiratory lumen, through which a breathing gas is supplied to a patient. The interior fins extend from an inner surface toward a central axis of the elongate conduit, while the exterior fins extend radially outwardly from an outer surface of the elongate shell.

### CROSS-REFERENCE TO RELATED APPLICATIONS

### STATEMENT RE: FEDERALLY SPONSORED RESEARCH/DEVELOPMENT

(Not Applicable)

### BACKGROUND OF THE INVENTION

The present invention relates generally to a heat exchanger, and more particularly, to an air-to-air heat exchanger that pre-cools the breathing gas output from a mechanical ventilator prior to delivery to a patient.

Humidification is required in the airway of a human body to maintain ciliary activity, prevent squamous epithelial changes (mucosal changes), dehydration and thickening of secretions and positive ETT obstruction, minimize atelectasis and tracheitis, and decrease heat loss. For a healthy human body, the heat transfer function of the nose adjusts the inspired gas to be approximately 36° C. and about 80% to 90% saturated with water vapor while reaching the carina. Without the equivalent heat transfer function, mouth breathing reduces the relative humidity to about 60% to 70%. Heat and moisture content falls from the carina to the nostrials, so that the nose temperature is typically 30° C. The countercurrent mechanism of heat and moisture exchange with nasal cooling on inspiration and warming on exhalation in the airway maximizes breathing efficiency.

When the upper airway of the human body is bypassed, that is, when a patient is breathing with the aid of a mechanical ventilator, humidification is required to prevent hypothermia, inspissation of airway secretions, destruction of airway epithelium and atelectasis as mentioned above. The humidification has been accomplished using a heated humidifier or a heat and moisture exchanger (HME). Conventional heated humidifiers are typically heavy, bulky, and therfore lack portability. In addition, such heated humidifiers operate actively to increase the heat and water vapor content of inspired gas. Under certain circumstance, water condensation may contaminate the supply path of the breathing air and/or interfere with the function of the ventilator.

Conventional heat and moisture exchangers, often referred as the hygroscopic condenser humidifiers, operate passively by storing heat and moisture from the exhaled gas, and releasing the heat and moisture to the inhaled gas. For conventional heat and moisture exchangers, moisture is limited by the gas exhaled from the patient. As such, when the temperature differential between the breathing air heated by the exhaled gas and the ambient temperature is small, the cooling effect of the breathing air is poor and results in insufficient moisture to properly desiccate the airway of the patient.

Therefore a substantial need exists in the art to provide efficient heat-to-moisture exchange, particularly when the temperature differential between the heated breathing air and the ambient temperature is relatively low, so as to provide sufficient moisture in the breathing air to the patient.

### BRIEF SUMMARY OF THE INVENTION

The present invention specifically addresses the above referenced need in the art by providing an air-to-air heat exchanger for pre-cooling and humidifying breathing gas supplied to a patient. The air-to-air heat exchanger comprises an elongate conduit or shell, a plurality of interior fins and a plurality of exterior fins. The elongate shell is preferably installed adjacent the output of an inspiratory lumen, through which breathing gas is supplied to a patient. The interior fins extend from an inner surface toward a central axis of the elongate shell, while the exterior fins extend radially outward from an outer surface of the elongate shell.

Preferably, the interior and exterior fins are made of low specific heat material such as aluminum and/or stainless steel to provide effective heat exchange for the breathing air flowing there through. Therefore, when the temperature of the breathing air is higher than the ambient temperature, the breathing air can be effectively cooled. In one embodiment of the present invention, the elongate shell includes a cylindrical shell.

The present invention further provides an air-to-air heat exchanger for pre-cooling and humidifying breathing gas supplied to a patient. The air-to-air heat exchanger comprises an elongate shell installed adjacent the output of an inspiratory lumen, through which breathing gas is supplied to a patient, a plurality of interior and exterior fins extending from an inner surface towards a central axis of the elongate conduit or shell, and a plurality of exterior fins extending radially from an outer surface of the elongate shell. In this embodiment, the air-to-air heat exchanger further comprises an external shield enclosing the elongate shell therein, and a cooling medium, preferably comprising an air source, connected to the external shield. The air source is operative to supply cooling air into the external shield for more effectively adjusting the temperature of the breathing gas.

The air-to-air heat exchanger comprising the external shield and the air source is particularly applicable when the ambient temperature is higher than the temperature of the breathing air flowing through the air-to-air heat exchanger. By supplying cool air, the elongate shell enclosed by the external shield can thus be effectively cooled without being affected by the ambient temperature, so that proper humidification can be achieved. Similarly, the elongate shell, the interior and exterior fins are preferably made of aluminum or stainless steel.

The present invention further provides a ventilator that incorporates an air-to-air heat exchanger. The ventilator comprises an expiratory circuit to which the patient exhales and an inspiratory circuit through which the breathing gas is supplied to the patient. The inspiratory circuit further comprises an oxygen supply, an air supply, a blender, a flow control valve, and an air-to-air heat exchanger. The oxygen and air supply are connected to one end of the inspiratory circuit for supplying oxygen and air to the patient. The oxygen and air are then mixed into breathing gas by the blender. The flow rate of the breathing gas is controlled by the flow control valve. The air-to-air heat exchanger is connected to or adjacent the flow control valve to adjust the temperature of the breathing gas. The air-to-air heat exchanger further comprising an elongate shell and a plurality of fins radially extending from a surface of the elongate shell.

In the above ventilator, the inspiratory circuit is preferably connected to the expiratory circuit to receive heat and moisture of the gas exhaled by the patient. The air-to-air heat exchanger is preferably made of aluminum or stainless steel, such that the breathing gas can be effectively cooled even when the temperature differential between the breathing gas and the environment is relatively small. The fins further comprise a plurality of interior fins extending toward a central axis of the elongate shell from an inner surface thereof and a plurality of exterior fins, extending radially outward from an outer surface of the elongate shell.

In one embodiment of the present invention, the ventilator further comprises an external shield enclosing the air-to-air heat exchanger therein and an air source operative to supply cooled air into the external shield to effectively adjust temperature of the breathing air flowing through the air-to-air heat exchanger.

### BRIEF DESCRIPTION OF THE DRAWINGS

These, as well as other features of the present invention, will become more apparent upon reference to the drawings wherein:

FIG. 1 comprises a perspective view of the air-to-air heat exchanger of the present invention;

FIG. 2 comprises a top view of the air-to-air heat exchanger;

FIG. 3 comprise a side view of the air-to-air heat exchanger;

FIG. 4 depicts a block diagram of an embodiment of ventilator according to the present invention; and

FIG. 5 depicts a block diagram of another embodiment of ventilator according to the present invention.

### DETAILED DESCRIPTION OF THE INVENTION

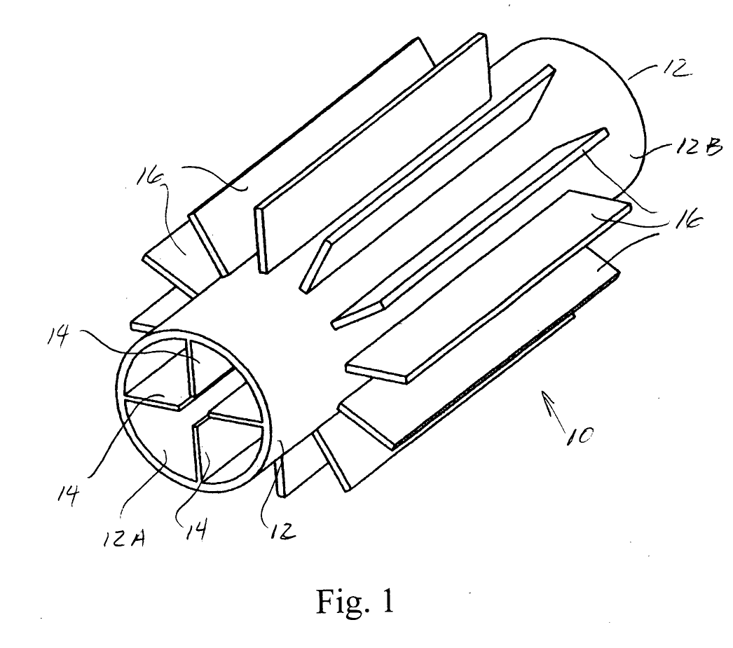

The present invention provides an air-to-air heat exchanger to be installed adjacent the output lumen of a medical ventilator to provide breathing air to a patient. The perspective view, the cross-sectional view and the side view of the air-to-air heat exchanger 10 are illustrated in FIGS. 1, 2 and 3, respectively. As shown in FIG. 1, the air-to-air heat exchanger 10 includes an elongate hollow annular tube or conduit 12, a plurality of interior fins 14 and a plurality of exterior fins 16. In this embodiment, the elongate conduit 12 is preferably formed in a cylindrical shape. It will be appreciated that according to specific geometry of the output lumen of the breathing air path, the shape of the elongate shell 12 can be altered as required.

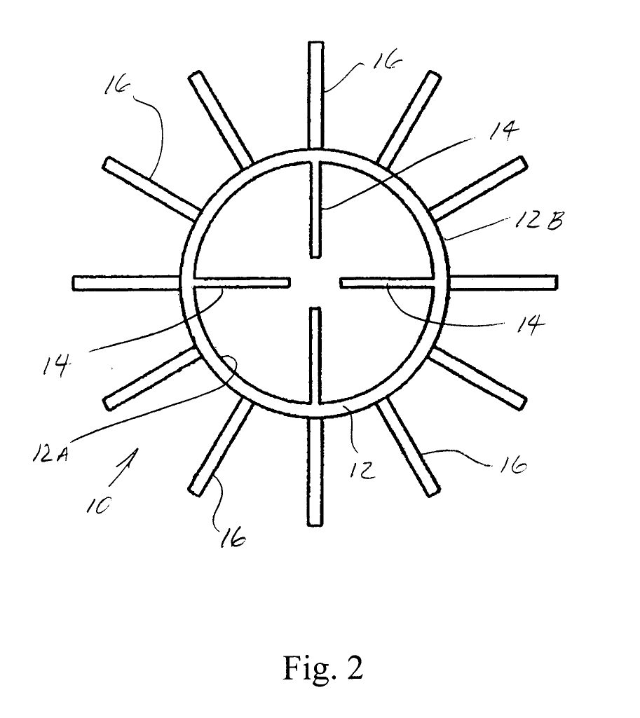

The interior fins 14 extend radially inwardly from the inner surface 12a of the elongate conduit 12, and the exterior fins 16 extend radially outward from an outer surface 12b of the elongate shell 12. Although not by way of limitation, in the embodiment as shown in FIG. 2, the interior fins 14 extend only partially radially inward toward the central axis of the elongate shell 12. As such the radial length of the interior fins 14 is preferably smaller than the inside radius of the elongate conduit 12 to thereby minimize flow restriction through the conduit 12. Referring to FIG. 1, the axial length of both the interior and exterior fins 14 and 16 are preferably shorter than the length of the elongate conduit 12 such that the conduit 12 can be easily installed in the inspiration circuit of the ventilator. The radial length of the exterior fins 14 may be varied according to desired heat transfer characteristics as well as the diameter/radius of the output lumen into which the air-to-air heat exchanger 10 is disposed. Further, in the embodiment as shown in FIGS. 1 and 2, the air-to-air heat exchanger 10 preferably includes four interior fins 14 and twelve exterior fins 16. However it will be appreciated that the number of the interior and exterior fins 14 and 16 can be varied according to specific geometry of the output lumen and the elongate shell 12, or other specific requirements.

As mentioned above, the heat transfer function of the nose effectively cools down the inspired gas, and it is known that the relative humidity is more easily saturated when the air is cooler. According to the relationship between the specific heat S, the temperature variation T and the heat H expressed as:

S∝H/mΔT,

to produce an effective heat transfer, the material for forming the elongate conduit 12, the exterior fins 14 and the interior fins 16 includes material with a relative low specific heat, such as aluminum and/or stainless steel. Therefore, when the heated breathing air flows through the air-to-air heat exchanger 10, the relatively low specific heat allows the air-to-air heat exchanger 10 to effectively absorb the heat from the breathing air and transfer the same via conduction and convection to the ambient environment. Consequently, the breathing air is effectively cooled to a temperature at which the relative humidity is increased.

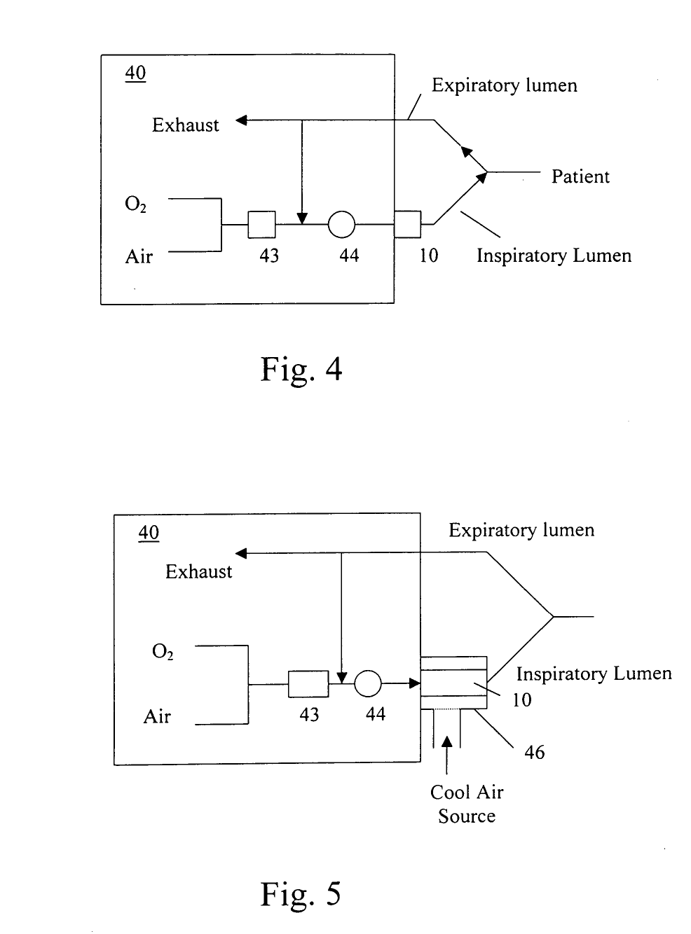

The air-to-air heat exchanger 10 can be applied to all kinds of ventilators, such that the breathing air can be adjusted with proper humidity before being supplied to the patient. FIG. 4 shows the exemplarily breathing circuit 40 that includes the air-to-air heat exchanger 10. In FIG. 4, the breathing circuit 40 comprises an expiratory gas lumen to which the patient exhales, and an inspiratory gas lumen through which the breathing gas is supplied to the patient. The distal end of the inspiratory gas lumen is connected to an air supply and optionally an oxygen supply. The oxygen and the air are then mixed and processed by a blender 43, through which the breathing gas with a specific oxygen content is provided. The inspiratory lumen 41 may further be connected to the expiratory gas lumen 41, such that the heat and moisture of the exhaled gas is delivered to the breathing gas.

The inspiratory lumen further includes a flow controller 44, which controls the flow rate of the breathing gas supplied to the patient in various breathing stages of the patient. The air-to-air heat exchanger 10 is installed adjacent the output of the inspiratory lumen 41, so as to be in series with the inspiratory circuit. As the air-to-air heat exchanger 10 is made of a material with a relatively low specific heat, as the breathing gas travels through the conduit 12 the heat of the breathing gas is absorbed by the internal fins 14 and transferred via conduction through the annular wall of the conduit 12 and into the external fins 16 and subsequently transferred via convection from the external fins 16 to the ambient environment. Consequently, the temperature of the breathing gas is lowered to increase the relative humidity thereof.

In one embodiment of the present invention, the flow controller 44 includes a drag compressor which accelerates the flow rate of the breathing gas during the inspiratory phase and decelerates or stops the flow rate of the breathing gas during the expiratory phase. The functions and structures of the ventilator 40 are disclosed in U.S. Pat. No. 5,868,133, assigned to the subject assignee the disclosure of which is expressly incorporated herein by reference. When the ambient environment of the ventilator 40 is maintained a relative low temperature, and the temperature of the breathing gas flowing through the controller 44 is substantially higher, the air-to-air heat exchanger 10 is operative to effectively lower the temperature of the breathing gas to a desired level.

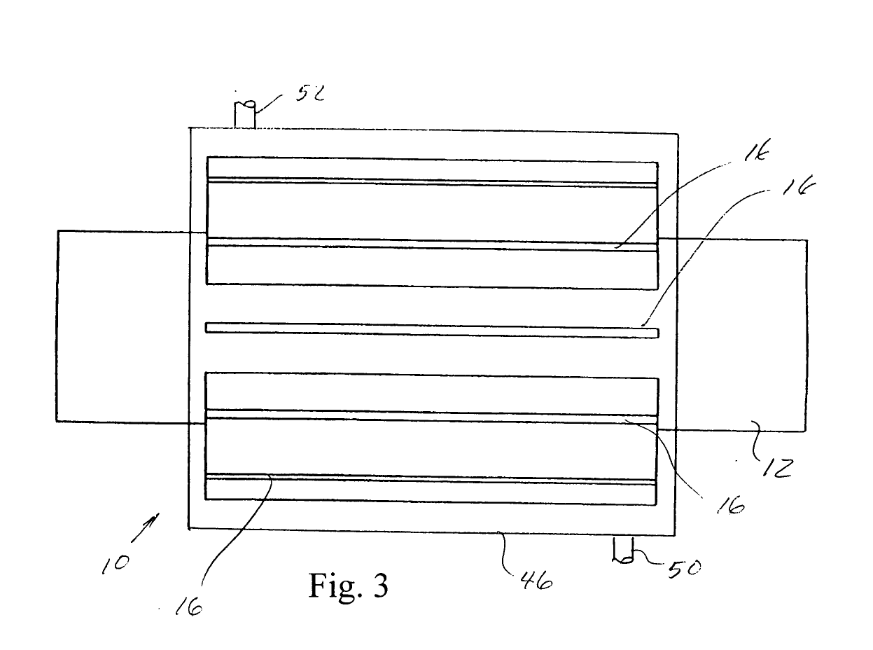

Under those situations where the temperature of the breathing gas is substantially higher than the ambient temperature on where the temperature of the ambient environment is high, as shown in FIGS. 3 and 5, the air-to-air heat exchanger 10 may be enclosed within an external housing or shield 46. The external shield 46 may be connected via inlet 50 (shown in FIG. 3) to a cool air source. As outlet 52 may additionally be provided on the shield. As cool air is directed into the shield 46 through the inlet 50 and outlet 52 the breathing air flowing through the air-to-air heat exchanger 10 can be effectively cooled and maintain proper humidity. Those having ordinary skill in the art will further recognize that the air source may be replaced with a water source or refrigerant source for greater heat transfer capability as desired.

Indeed, each of the features and embodiments described herein can be used by itself, or in combination with one or more of other features and embodiment. Thus, the invention is not limited by the illustrated embodiment but is to be defined by the following claims when read in the broadest reasonable manner to preserve the validity of the claims.

What is claimed is:

1. An air-to-air heat exchanger for pre-cooling and humidifying a breathing gas supplied to a patient, comprising:
an elongate conduit, installed adjacent the output of an inspiratory lumen, through which a breathing gas is supplied to a patient;
at least one interior fin, extending from an inner surface towards a central axis of the elongate conduit; and
at least one exterior fin, extending outwardly from an outer surface of the elongate conduit.

2. The air-to-air heat exchanger according to claim 1, wherein the elongate conduit and at least one of the inner and outer fins are made of aluminum.

3. The air-to-air heat exchanger according to claim 1, wherein the elongate conduit includes an annular configuration.

4. An air-to-air heat exchanger for pre-cooling and humidifying a breathing gas supplied to a patient, comprising:
an elongate conduit, installed adjacent the output of an inspiratory lumen, through which a breathing gas is supplied to a patient;
a plurality of interior fins, extending from an inner surface towards a central axis of the elongate conduit;
a plurality of exterior fins, extending outwardly from
an outer surface of the elongate conduit;
an external shield, enclosing the elongate conduit therein; and
an inlet and outlet formed on said external shield for directing an air source through the external shield.

5. The air-to-air heat exchanger according to claim 4, wherein the inlet and outlet are operative to supply the air source into the external shield for adjusting temperature of the breathing gas.

6. The air-to-air heat exchanger according to claim 4, wherein the air source comprises a cool air source and the inlet and outlet are operative to supply a cool air into the external shield for cooling the breathing gas.

7. The air-to-air heat exchanger according to claim 4, wherein the elongate conduit and the inner and outer fins are made of aluminum.

8. The air-to-air heat exchanger according to claim 4, wherein the elongate conduit includes a cylindrical cross-sectional configuration.

9. A ventilator, comprising:
an expiratory circuit, to which a patient exhales a gas;
an inspiratory circuit, from which a breathing gas is supplied to the patient, the inspiratory circuit further comprising:
an oxygen supply, operative to supply an oxygen source;
an air supply, operative to supply an air source to the patient;
a blender, connected to the oxygen supply and the air supply to produce a breathing gas;
a flow controller, connected to the blend to control flow rate of the breathing gas; and
an air-to-air heat exchanger, connected to the flow controller to adjust temperature of the breathing gas; wherein the air-to-air heat exchanger further comprising:
an elongate conduit; and
a plurality of fins radially extending from a surface of the elongate shell.

10. The ventilator according to claim 9, wherein the inspiratory circuit is connected to the expiratory circuit to receive heat and moisture of the gas exhaled by the patient.

11. The ventilator according to claim 9, wherein the air-to-air heat exchanger is made of aluminum.

12. The ventilator according to claim 9, wherein the fins further comprise:
a plurality of interior fins, extending towards a central axis of the elongate shell from an inner surface thereof; and
a plurality of exterior fins, extending outwardly from an outer surface of the elongate shell.

13. The ventilator according to claim 11, further comprising:
an external shield enclosing the air-to-air heat exchanger therein; and
an air source, operative to supply an air into the external shield to adjust temperature of the breathing air flowing through the air-to-air heat exchanger.

14. The ventilator according to claim 13, wherein the air source is operative to supply cool air for cooling the breathing air.
